(19) United States
(12) Patent Application Publication
(10) Pub. No.: US 2006/0095117 A1
(43) Pub. Date: May 4, 2006

(54) Apparatus and method for temporarily clamping a tubular graft to a prosthetic cardiac valve

(76) Inventors: Carl F. Popelar, Austin, TX (US); Brant H. Maines, Colleyville, TX (US); Norma I. Gonzalez, Austin, TX (US); David W. Mester, Austin, TX (US)

Correspondence Address: WILLIAMS, MORGAN & AMERSON, 10333 RICHMOND, SUITE 1100, HOUSTON, TX, 77042, US

(21) Appl. No.: 10/980,640

(22) Filed: Nov. 3, 2004

(51) Int. Cl. A61F 2/24; A61F 2/06

(52) U.S. Cl. 623/1.26; 623/1.36; 623/2.4; 623/2.11; 606/153

(57) ABSTRACT

An apparatus for temporarily clamping a tubular graft to a prosthetic cardiac valve includes a tube having a proximal end and a distal end and a clamping line partially disposed within the tube and having a secured end fastened to the tube and a free end extending from the tube. The clamping line forms a loop extending from the proximal end of the tube that is adapted to expand as the free end is advanced into the tube and is contracted as the free end is withdrawn from the tube.

### BACKGROUND OF THE INVENTION

1. Field of the Invention

This invention generally relates to the field of cardiac repair and, in particular, an apparatus and method for temporarily clamping a tubular graft to a prosthetic cardiac valve.

2. Description of the Related Art

In the mammalian heart, deoxygenated blood flows into the right atrium through the superior vena cava and the inferior vena cava. Upon contraction of the right atrium, the deoxygenated blood flows into the right ventricle. When the right ventricle contracts, the deoxygenated blood is pumped through the pulmonary artery to the lungs. Oxygenated blood returning from the lungs enters the left atrium. From the left atrium, the oxygenated blood flows into the left ventricle, which in turn pumps oxygenated blood to the body via the aorta and lesser arteries branching from the aorta.

This pumping action is repeated in a rhythmic cardiac cycle in which the ventricular chambers alternately contract and pump, then relax and fill. As is well known, a series of one-way cardiac valves prevent backflow of the blood as it moves through the heart and the circulatory system. Between the atrial and ventricular chambers in the right and left sides of the heart are the tricuspid valve and the mitral valve, respectively. At the exits of the right and left ventricles are the pulmonic and aortic valves, respectively.

It is well known that various heart diseases may result in disorders of the cardiac valves. For example, diseases such as rheumatic fever can cause the shrinking or pulling apart of the valve orifice, while other diseases may result in endocarditis, an inflammation of the endocardium (membrane lining the heart). Resulting defects in the valves hinder the normal functioning of the atrioventricular orifices and operation of the heart. More specifically, defects such as the narrowing of the valve opening (valvular stenosis) or the defective closing of the valve (valvular insufficiency) result in an accumulation of blood in a heart cavity or regurgitation of blood past the valve. If uncorrected, prolonged valvular stenosis or valvular insufficiency can cause damage to the heart muscle, which may eventually necessitate total valve replacement.

These defects may be associated with any of the cardiac valves, although they occur most commonly in the left side of the heart. For example, if the aortic valve between the left ventricle and the aorta narrows, blood will accumulate in the left ventricle. Similarly, in the case of aortic valve insufficiency, the aortic valve does not close completely, and blood in the aorta flows back past the closed aortic valve and into the left ventricle when the ventricle relaxes.

In many cases, complete valve replacement is required. Mechanical artificial heart valves for humans are frequently fabricated from titanium, pyrolytic carbon, polymers or biologic tissue, including tissue from cattle, swine, or human. Such valves have become widely accepted and used by many surgeons.

Mechanical prosthetic heart valves typically comprise a rigid orifice supporting one, two or three rigid occluders, or leaflets. The occluders pivot between open and shut positions and thereby control the flow of blood through the valve. The orifice and occluders are commonly formed of pyrolytic carbon, which is a particularly hard and wear-resistant form of carbon. To minimize deflection of the orifice and possible interference with the movement of the occluders, the orifice is often surrounded by a stiffening ring, which may be made of titanium, cobalt chromium, or stainless steel. In one valve configuration, the orifice and stiffening ring are captured within a knit fabric sewing or suture cuff. This prosthetic valve is placed into the valve opening and the sewing cuff is sutured to the patient's tissue. Over time, tissue grows into the fabric of the cuff, providing a secure seal for the prosthetic valve.

However, in many patients, once degeneration of a valve has occurred, the surrounding blood vessels may also become diseased. Particularly in the case of the aortic valve, surgeons have found that the portion of the aorta adjacent to the valve is often degenerated to the degree that it must be replaced. Consequently, both the aortic valve and a segment of the ascending aorta may be replaced at the same time. When this technique was being developed, the surgeon would stitch a segment of vascular graft to the sewing ring of the mechanical valve after implanting the mechanical heart valve. However, this required a relatively long duration of surgery and was complicated to complete, potentially being deleterious to the well being of the patient.

The present invention is directed to overcoming, or at least reducing, the effects of one or more of the problems set forth above.

### SUMMARY OF THE INVENTION

In one aspect of the present invention, an apparatus for temporarily clamping a tubular graft to a prosthetic cardiac valve is provided. The apparatus comprises a tube having a proximal end and a distal end and a clamping line partially disposed within the tube and having a secured end fastened to the tube and a free end extending from the tube. The clamping line forms a loop extending from the proximal end of the tube that is adapted to expand as the free end is advanced into the tube and is contracted as the free end is withdrawn from the tube.

In another aspect of the present invention, a cardiac repair assembly is provided. The assembly includes a tubular graft comprising a skirt and a prosthetic cardiac valve comprising a sewing cuff, the prosthetic cardiac valve being disposed within the tubular graft such that the skirt is proximate the sewing cuff. The assembly further includes a tube having a proximal end and a distal end and a clamping line partially disposed within the tube and having a secured end fastened to the tube and a free end extending from the tube. The clamping line forms a loop extending around the skirt from the proximal end of the tube, such that the clamping line is tensioned to clamp the skirt to the sewing cuff.

In yet another aspect of the present invention, a method for temporarily clamping a tubular graft to a prosthetic cardiac valve is provided. The method includes providing a clamp comprising a tube having a proximal end and a distal end and a clamping line partially disposed within the tube and having a secured end fastened to the tube and a free end extending from the tube. The clamping line forms a loop extending from the proximal end of the tube that adapted to expand as the free end is advanced into the tube and is contracted as the free end is withdrawn from the tube. The method further includes placing the loop around a skirt of the tubular graft, inserting the prosthetic cardiac valve into the tubular graft such that the skirt covers a sewing cuff of the prosthetic cardiac valve, and tensioning the clamping line to clamp the skirt to the sewing cuff.

### BRIEF DESCRIPTION OF THE DRAWINGS

The invention may be understood by reference to the following description taken in conjunction with the accompanying drawings, in which the leftmost significant digit(s) in the reference numerals denote(s) the first figure in which the respective reference numerals appear, and in which:

FIG. 1 is a side, elevational view of an illustrative embodiment of an apparatus for temporarily clamping a tubular graft to a prosthetic cardiac valve according to the present invention;

FIG. 2 is an enlarged view of a distal end of the apparatus of FIG. 1 illustrating a friction anchor according to the present invention for retaining a free end of a clamping line;

FIG. 3 is a cross-sectional view of one particular embodiment of the clamping line of FIG. 1 and FIG. 2 taken along the line 3-3 of FIG. 2;

FIG. 4A-FIG. 4D are stylized diagrams depicting an illustrative embodiment of a method of using the apparatus of FIG. 1 according to the present invention;

FIG. 5 is a stylized diagram of the apparatus of FIG. 1 further including a graft stabilization loop according to the present invention;

FIG. 6 is an enlarged view of a portion of the apparatus of FIG. 4 illustrating retention knots in the graft stabilization loop; and

FIG. 7-FIG. 8 are stylized diagrams of alternative illustrative embodiments of an apparatus for temporarily clamping the tubular graft and the prosthetic cardiac valve in which tubes thereof are deformable to attain a desired shape.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present invention relates to an apparatus and method for temporarily clamping a tubular graft to a prosthetic cardiac valve. The tubular graft and prosthetic cardiac valve may then be placed in position for surgical implantation or they may be sutured or “tagged” together and then placed in position for implantation. These operations may be performed prior to placing the patient on a heart-lung machine, thus often reducing the amount of time the patent is attached to the heart-lung machine. Use of the present invention provides a convenient way to handle the graft and prosthetic valve as a single unit and allows both to be implanted simultaneously.

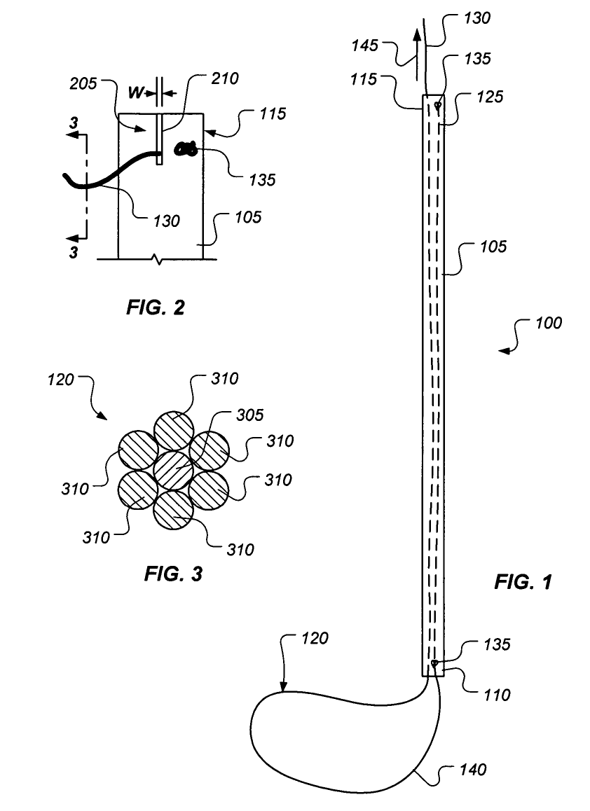

FIG. 1 depicts an illustrative embodiment of a clamp 100 for temporarily clamping a tubular graft to a prosthetic cardiac valve. In the illustrated embodiment, the clamp 100 comprises a tube 105 comprising a rigid or semi-rigid, biocompatible material such as polysulfones (e.g., Udel™ or Radel™), polyetherimide (e.g., Ultem™), acetals (e.g., Delrin™ or Celcon™), silicone elastomers, nitinol, or stainless steels and having a proximal end 110 and a distal end 115. A clamping line 120 is partially disposed within the tube and includes a secured end 125 and a free end 130. The secured end 125 is attached to the tube 105 and, in the illustrated embodiment, the secured end 125 includes a knot 135 for retaining it to the tube 105. The knot 135 may be disposed, for example, near the proximal end 110 or near the distal end 115. Note that FIG. 1 illustrates both locations for the knot 135. If the knot 135 is disposed near the proximal end 110, the clamping line 125 above the knot 135 will generally be omitted. The clamping line 125 forms a loop 140 extending from the proximal end 110 of the tube 105, with the free end 130 of the clamping line 120 extending from the distal end 115 of the tube 105. As the free end 130 of the clamping line 120 is withdrawn from the tube 105 (as indicated by an arrow 145), the loop 140 is contracted. Conversely, when the free end 130 is allowed to advance into the tube 105 (e.g., by pulling on the loop 140), the loop 140 is expanded.

As will be discussed below, it is often desirable to maintain the loop 140 at a particular size. Accordingly, as depicted in FIG. 2, one particular embodiment of the clamp 100 includes a friction anchor 205 for retaining the clamping line 120 at a desired position. In the illustrated embodiment, the friction anchor 205 comprises a slot 210 defined by the distal end 115 of the tube 105. The slot 210 has a width W that is smaller than a diameter of the clamping line 120. When at the desired position, the free end 130 of the clamping line 120 is urged into the slot 210, which deforms the free end 130, thus retaining it in the slot 210.

In various embodiments, the clamping line 120 may take on many forms, e.g., suture material, a thread, a string, a cord, a strip, a band, a ribbon, or the like. The clamping line 120 may comprise a single strand or be multistranded. In one embodiment, illustrated in FIG. 3, the clamping line 120 comprises a core 305 made of a higher strength or higher stiffness material, such as a metal, surrounded by a plurality of textile yarns 310 to form a stiffened line. In various embodiments, the clamping line comprises textile yarns such as braided polyester suture, non-absorbable monofilament polypropylene, or polytetrafluoroethylene (PTFE) fibers.

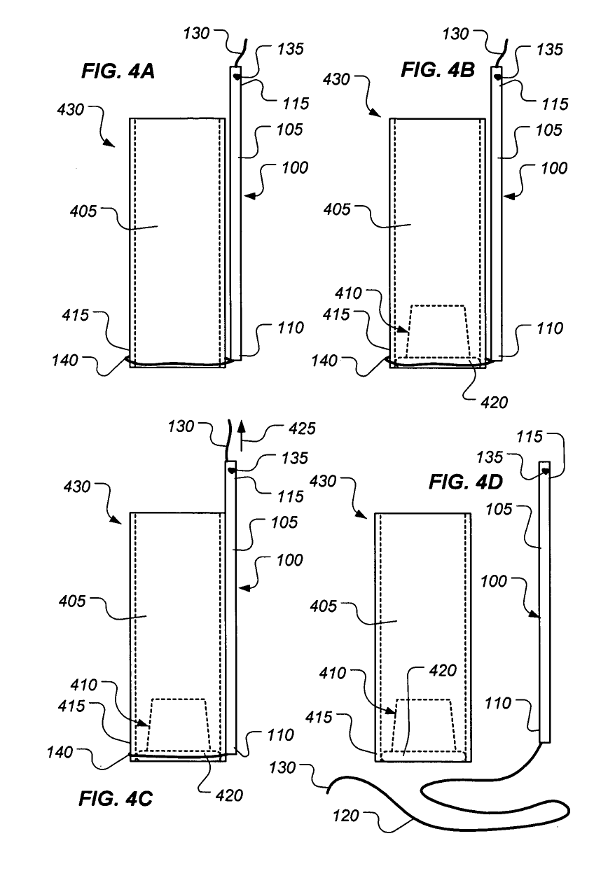

FIG. 4A-FIG. 4D illustrate one particular method of using the clamp 100 to temporarily clamp a tubular graft 405 to a prosthetic cardiac valve 410. The loop 140 of the clamping line 120 is sized to fit over the tubular graft 405 by advancing the free end 130 of the clamping line 120 into or withdrawing it from the tube 105, as discussed above. As depicted in FIG. 4A, the loop 140 is then placed over a proximal end or attachment skirt 415 of the tubular graft 405. The prosthetic cardiac valve 410 is inserted into the tubular graft 405 such that the attachment skirt 415 covers a sewing cuff 420 of the prosthetic cardiac valve 410, as shown in FIG. 4B. Referring to FIG. 4C, tension is applied to the free end 130 of the clamping line 120 (as indicated by an arrow 425) to tighten the loop 140 around the skirt 415 of the tubular graft 405, thus clamping the tubular graft 405 to the prosthetic cardiac valve 410.

The free end 130 of the clamping line 120 may be retained by the friction anchor 205, as described above and shown in FIG. 2, to maintain tension on the clamping line 120 and clamping of the tubular graft 405 to the prosthetic cardiac valve 410. The scope of the present invention, however, encompasses the use of other ways of retaining the free end 130 of the clamping line 120, such as by using a surgical clamp or retaining the free end 130 by hand.

The tubular graft 405, the prosthetic cardiac valve 410, and the clamp 100 clamping the tubular graft 405 to the prosthetic cardiac valve 410 comprise a cardiac repair assembly 430 according to the present invention. The cardiac repair assembly 430 may be immediately manipulated by the surgeon into position for implantation into the patient. Alternatively, it may be desirable to stitch or “tag” the tubular graft 405 to the prosthetic cardiac valve 410 to provide additional stability. In either case, once the tubular graft 405 and the prosthetic cardiac valve 410 have been implanted, the clamp 100 may be removed by releasing the free end 130 and moving the tube 105 away from the tubular graft 405, as shown in FIG. 4D. These actions cause the free end 130 of the clamping line 120 to move through the tube 105 and exit the proximal end 110 thereof, thus releasing the loop 140 from the tubular graft 405. Even in this configuration, the clamping line 120 is retained with the tube 105 and can be removed from the surgical area as a unit.

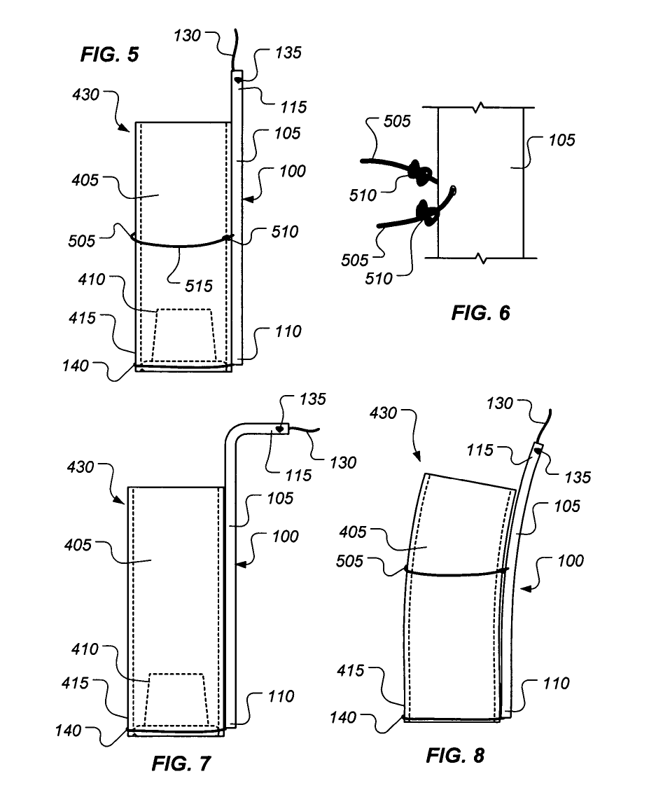

Depending upon the length of the tubular graft 405, it may be desirable to further stabilize the graft 405 with respect to the tube 105. Thus, as illustrated in FIG. 5, the clamp 100 may further include a graft stabilization loop 505 attached to the tube 105. As the graft stabilization loop 505 is used to stabilize the tubular graft 405 with respect to the tube 105, rather than to clamp the tubular graft 405 to another member, the graft stabilization loop 505 may, in certain embodiments, be preformed, presized, and nonadjustable. In the illustrated embodiment, therefore, the graft stabilization loop 505 is not tightened against the tubular graft 405, providing some slack in the graft stabilization loop 505. In the illustrative embodiment shown in FIG. 6, the graft stabilization loop 505 extends through the tube 105. The graft stabilization loop 505 may, in various embodiments, comprise any of the forms or materials comprising the clamping line 120 or other suitable forms or materials.

After the tubular graft 405 and the prosthetic cardiac valve 410 have been implanted into the patient, the graft stabilization loop 505 may be cut by any desired means between knots 510 (only one shown in FIG. 5) and be retained on the tube 105. For example, if the graft stabilization loop 505 is cut between the knots 510 generally at 515, the graft stabilization loop 505 will remain attached to the tube 105, lessening the likelihood of errant materials being left in the surgical area.

It may also be desirable in certain situations to reconfigure the clamp 100 so that the tubular graft 405 and the prosthetic cardiac valve 410 may be manipulated into difficult to reach locations. For example, in the illustrative embodiment shown in FIG. 7, the tube 105 comprises a deformable, reconfigurable material, such as stainless steel or nitinol, or a polymeric or elastomeric tube with a deformable, reconfigurable core material (e.g., stainless steel or nitinol), which allows it to be bent into a more convenient shape for manipulating the tubular graft 405 and the prosthetic cardiac valve 410. Further, as illustrated in FIG. 8, the tube 105 may be deformed to urge the tubular graft 405 into a particular shape to aid in implantation.

Note that the tubular graft 405 and the prosthetic cardiac valve 410 in FIG. 3A-FIG. 8 are stylized depictions and are not intended to limit the present invention in any way. Rather, the scope of the present invention includes the use of the clamp 100 in conjunction with any type of tubular graft and prosthetic cardiac valve, comprising natural and/or man-made materials, and having various configurations as are known to the art.

This concludes the detailed description. The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed is:

1. An apparatus for temporarily clamping a tubular graft to a prosthetic cardiac valve, comprising:
a tube having a proximal end and a distal end; and
a clamping line partially disposed within the tube and having a secured end fastened to the tube and a free end extending from the tube, such that the clamping line forms a loop extending from the proximal end of the tube that is adapted to expand as the free end is advanced into the tube and is contracted as the free end is withdrawn from the tube.

2. An apparatus, according to claim 1, wherein the tube comprises a friction anchor for temporarily retaining the free end of the clamping line at a desired position.

3. An apparatus, according to claim 2, wherein the friction anchor comprises a slot defined by the distal end of the tube and having a width smaller than a diameter of the free end of the clamping line.

4. An apparatus, according to claim 1, further comprising a graft stabilization loop attached to the tube.

5. An apparatus, according to claim 4, wherein the graft stabilization loop extends through the tube and comprises a pair of knots, such that the tube is disposed between the pair of knots.

6. An apparatus, according to claim 4, wherein the graft stabilization loop comprises a stiffened line.

7. An apparatus, according to claim 1, wherein the clamping line comprises a stiffened line.

8. An apparatus, according to claim 1, wherein the tube is deformable.

9. A cardiac repair assembly, comprising:
a tubular graft comprising a skirt;
a prosthetic cardiac valve comprising a sewing cuff, the prosthetic cardiac valve being disposed within the tubular graft such that the skirt is proximate the sewing cuff;
a tube having a proximal end and a distal end; and
a clamping line partially disposed within the tube and having a secured end fastened to the tube and a free end extending from the tube, the clamping line forming a loop extending around the skirt from the proximal end of the tube, such that the clamping line is tensioned to clamp the skirt to the sewing cuff.

10. An apparatus, according to claim 9, wherein the tube comprises a friction anchor for temporarily retaining the free end of the clamping line to maintain a desired tension in the clamping line.

11. An apparatus, according to claim 10, wherein the friction anchor comprises a slot defined by the distal end of the tube and having a width smaller than a diameter of the free end of the clamping line.

12. An apparatus, according to claim 9, further comprising a graft stabilization loop attached to the tube.

13. An apparatus, according to claim 12, wherein the graft stabilization loop extends through the tube and comprises a pair of knots, such that the tube is disposed between the pair of knots.

14. An apparatus, according to claim 12, wherein the graft stabilization loop comprises a stiffened line.

15. An apparatus, according to claim 9, wherein the clamping line comprises a stiffened line.

16. An apparatus, according to claim 9, wherein the tube is deformable.

17. A method for temporarily clamping a tubular graft to a prosthetic cardiac valve, comprising:
providing a clamp comprising a tube having a proximal end and a distal end and a clamping line partially disposed within the tube and having a secured end fastened to the tube and a free end extending from the tube, such that the clamping line forms a loop extending from the proximal end of the tube that is adapted to expand as the free end is advanced into the tube and is contracted as the free end is withdrawn from the tube;
placing the loop around a skirt of the tubular graft;
inserting the prosthetic cardiac valve into the tubular graft such that the skirt covers a sewing cuff of the prosthetic cardiac valve; and
tensioning the clamping line to clamp the skirt to the sewing cuff.

18. A method, according to claim 17, further comprising anchoring the free end of the clamping line to maintain tension in the clamping line.

19. A method, according to claim 17, further comprising releasing the tension on the clamping line after surgically implanting the tubular graft and the prosthetic cardiac valve.

20. A method, according to claim 17, wherein tensioning the clamping line further comprises withdrawing the free end of the clamping line from the distal end of the tube.

21. A method, according to claim 17, further comprising placing a graft stabilization loop of the clamp around the tubular graft.
